(19) United States
(12) Patent Application Publication
(10) Pub. No.: US 2005/0239209 A1
(43) Pub. Date: Oct. 27, 2005

(54) Method for peptide and polypeptide purification and differential analysis

(76) Inventors: Jonathan Krakover, Dublin, PA (US); Ramila Philip, Warminster, PA (US)

Correspondence Address: FOX ROTHSCHILD O'BRIEN & FRANKEL LLP;PRINCETON PIKE CORPORATE CENTER, 997 LENOX DRIVE, BUILDING 3, LAWRENCEVILLE, NJ, 08648, US

(21) Appl. No.: 11/112,896

(22) Filed: Apr. 22, 2005

Related U.S. Application Data
(60) Provisional application No. 60/564,901, filed on Apr. 23, 2004.

(51) Int. Cl. C12P 21/06; G01N 33/00

(52) U.S. Cl. 436/86; 530/333; 530/409

(57) ABSTRACT

A method for peptide and polypeptide purification that uses a derivatized solid support to capture all of the polypeptides and peptides present in a solution and in a subsequent step quantitatively release all of the polypeptides and peptides bound to the solid support is disclosed. This method provides for quantitative recovery and permits further analysis by any of a variety of analytical methods, including sequencing, chromatography, mass spectrometry and biological assay. The method may further be used for screening peptides and polypeptides that bind to organic molecules such as small molecule drugs. The method does not irreversibly add any unwanted label moieties or otherwise alter the peptides during the release process that could interfere with subsequent processing or analysis steps, in particular, biological assay to assess function in vivo or in vitro in cell-based, whole animal or human testing.

### FIELD OF THE INVENTION

This invention relates generally to proteomics and more specifically to methods for peptide and polypeptide purification, sequence analysis and quantitation of peptides and polypeptides. Also provided are methods for modifying peptides or polypeptides in a sample for the differential analysis of two or more polypeptide samples from different sources. The polypeptides may be extracted from biological sources or may be chemically synthesized.

### BACKGROUND OF THE INVENTION

The proteins expressed in living cells and tissues define the physiological, developmental or health status of the cell or tissue at that time. When analyzing complex mixtures of proteins and peptides in cells, biological fluids or protein complexes, it is critical to assure that all species of interest are captured and carried through the extraction and purification processes for later analysis. This is especially critical for proteomics studies, where the total complement of proteins or peptides in cellular samples must be retained during isolation and characterization steps to ensure a thorough and systematic analysis. Additionally, it is desirable when performing comparative studies to be able to discern which proteins or peptides originated within different samples so that the compositions of those samples can be compared, with the assurance that the total complement of proteins and peptides is retained during purification, which is vital to a complete and meaningful analysis.

The quantitative and qualitative differences between protein profiles of the same cell type in different states can be used to understand the transitions between respective states. Examples include differential patterns of protein expression in different tissues, different organisms, different developmental stages, in health and disease, including cancers, autoimmune disorders and bacterial and viral infections. For example, proteomics can involve comparing the proteins present in a diseased cell to those in a non-diseased cell to identify disease-specific proteins. Proteomics is expected to greatly boost the number of novel drug targets that are of interest for the development of new drugs. Other applications include metabolic pathway analysis, study of protein interactions and disease diagnosis. Patterns of differential gene and protein expression can be used for diagnostic purposes, to indicate the presence of disease, for example, cancer, viral or bacterial infection. Certain proteins and peptides have direct applications as immunotherapeutics, diagnostics, theranostics, and drug and antibody targets.

Ideally, the method of purification and quantitation of complex mixtures of proteins and peptides will not irreversibly modify them during the purification and analysis processes. This is particularly important when isolating and characterizing unknown proteins and peptides that are naturally post-translationally modified (e.g. glycosylated, phosphorylated), because modification of the natural protein or peptide may diminish or alter the biological activity being evaluated, for example, in evaluation as biotherapeutic, immunotherapeutic or other types of drug candidates.

Particularly challenging is the purification and identification of short (˜6 amino acid) peptides. Short peptides are readily lost during purification processes because they typically do not possess structural or physical properties that allow them to be quantitatively captured and purified by existing methods. Therefore, there is a need for a method that quantitatively captures even very short peptides. Of particular interest is the quantitative capture and purification of Major Histocompatibility Complex (MHC)-associated Class I and Class II peptides that play an important role in the understanding of immune system function in health and disease and may be used for diagnostic, theranostic, or immunotherapeutic purposes. A minimum of six amino acids is needed to use a peptide to identify its protein of origin and gene sequence by gene or protein sequence database searching, however no methods currently exist to quantitatively capture peptides of this length using existing purification methods.

Numerous methods for protein purification have been developed based upon specific physical/chemical or structural properties of peptides or proteins. Examples include affinity separation based on specific group or groups (sulfhydryl groups, avidin/biotin labeling, Protein A, immunoprecipitation), amino acid composition (aromatic or charged amino acids), solubility, ionic and hydrogen bonding properties, and three dimensional structure. In each case, proteins or peptides that do not have the property essential to being captured by that specific procedure will be lost during the purification process.

A variety of existing methods for peptide and polypeptide purification require the addition of modifying groups to facilitate purification. Limitations of such methods are irreversible addition of a chemical reagent to the polypeptide, or the requirement for specific amino acids or chemically reactive groups (e.g., hydroxyl, carboxyl) groups to be present in order to attach the label to the polypeptide. Such residues are unlikely to be present on polypeptides of ten amino acids or less in length, and would not be captured by such modifying reagents.

U.S. Pat. No. 6,379,971 describes a method for attaching a “unique mass tag” label onto a peptide for the purpose of gaining sequence information. This “unique mass tag” label is covalently bound to the polypeptide and cannot be removed without destroying the polypeptide. The present method does not attach a covalent label to the polypeptide and therefore does not modify the polypeptide. U.S. Pat. No. 6,379,971 does not suggest any means for polypeptide purification.

U.S. Pat. No. 5,534,440 describes a method for attaching a label to a peptide via isothyocyanate (ITC) chemistry, and this label also functions as an aid in ionization for mass spectrometry analysis. U.S. Pat. No. 5,534,440 only describes a method for enhancing analysis and does not suggest a method for peptide or protein purification.

U.S. Patent application 20020168644 describes a method for labeling a peptide with a “cleavable functional group” label, however, after cleavage, a portion of the label still is attached to the peptide.

U.S. Patent application 20020076739 describes a method for isotopically labeling two or more different samples, however, the sequence A-L-PRG is required where A is an affinity label that selectively binds to a capture reagent, L is a linker group which can be differentially labelled with stable isotopes and PRG is a protein reactive group that selectively reacts with certain protein functional groups. The present invention does not require any specific structures or residues to be present in the polypeptide in order for isotopic labeling to be carried out.

U.S. Patent application 20030017507 describes a method for differential labeling of polypeptides using isotopes using a reaction dependent upon dithiol addition. The present invention is based on isothyocyanate (ITC) chemistry, not dithiol chemistry.

### SUMMARY OF THE INVENTION

In one aspect, this invention provides a method for peptide and polypeptide purification that uses a derivatized solid support to capture all of the polypeptides and peptides present in a solution and in a subsequent step quantitatively release all of the polypeptides and peptides bound to the solid support. This method provides for quantitative recovery and permits further analysis by any of a variety of analytical methods, including sequencing, chromatography, mass spectrometry and biological assay. The method may further be used for screening peptides and polypeptides that bind to organic molecules such as small molecule drugs. The method does not irreversibly add any unwanted label moieties or otherwise alter the peptides during the release process that could interfere with subsequent processing or analysis steps, in particular, biological assay to assess function in vivo or in vitro in cell-based, whole animal or human testing.

In another aspect, this invention provides a procedure for end-labeling every peptide or polypeptide chain in a mixture in the process of releasing the peptides and polypeptides from a solid support. The invention further provides for a method to detach every polypeptide chain from the solid support without adding any modifying groups to the polypeptide.

The method further provides a process for differentially labeling all of the peptides or polypeptides in a mixture without altering their biological properties as a means for later identifying the source of the peptides or polypeptides when they are mixed with peptides or polypeptides from a different source.

Therefore this method provides a novel use for isothiocyanate derivatives attached to solid supports in peptide and polypeptide isolation and purification and differentiation of peptides and polypeptides obtained from different sources. More specifically, this method is useful for isolation and quantitative end-labeling of peptides of at least two amino acids in length, and most preferably for peptides amino acids of 8-10 amino acids in length, as well as for isolation and quantitative end-labeling of polypeptides equal to or greater than 10 amino acids in length. The method does not alter or interfere with or modify any post-translational modifications present on the peptides or polypeptides. In addition, this method is applicable to any molecule that presents a peptide-like structure.

The method further provides for a means to purify synthetic oligomers and polymers having the structure: 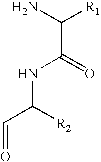
where R1 and R2may contain aliphatic groups, aromatic groups or inorganic groups. The method further provides for a means to purify organic molecule drugs or molecules with drug-like properties, where R1 and R2 may contain aliphatic or aromatic groups or inorganic groups, for example steroidal compounds.
Definitions

Peptide—A chain of amino acids joined by a peptide bond of 2 to 10 amino acids in length.

Polypeptide—A protein or part of a protein made of a chain of amino acids joined by a peptide bond containing 10 to more than 100 amino acids.

### DETAILED DESCRIPTION OF THE INVENTION

The present invention uses isothiocyanate chemistry, more specifically diisothiocyanate (DITC), a derivative of Edman's reagent and chemically reactive to primary amines, and attached to a solid support, such as glass, as a means to quantitatively capture and end-label polypeptides at their amino terminus. Any isothiocyanate derivative may be used, including, but not limited to PITC (phenylisothiocyanate), p-phenylene diisothiocyanate, rhodamine B isothiocyanate, 4-(4-isothiocyanatophenylazo)-N,N-dimethylaniline. DITC glass was developed to facilitate peptide sequencing because it quantitatively and covalently binds primary amines such as those on the termini of polypeptides. When DITC is bound to a solid support such as glass, polypeptides and other labeled molecules having free amino termini are quantitatively and covalently bound to the DITC glass. The sample can be thoroughly washed to remove contaminants before the bound polypeptides of length n−1 are removed using an acidic reagent, most preferably trifluoroacetic acid (TFA), however any organic acid can be used. DITC glass is commonly used in peptide sequencing, however there has been no suggestion of using DITC glass as a reagent for the quantitative capture, purification and differential labeling of peptides, polypeptides, polymers or other synthetic or naturally occurring organic molecules having a single terminal primary amine and the structure:

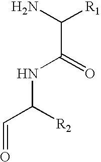

In one embodiment of the invention in which a peptide or polypeptide is purified from a complex mixture, a peptide- or polypeptide-containing sample is dissolved in a buffer of 5% triethylamine in 50/50 water/isopropanol and incubated with DITC (diisothiocyanate) glass (G9764; Sigma-Aldrich; St. Louis, Mo.) for most preferably 45 minutes, however any interval between 30 to 60 minutes can also be used. The incubation should take place most preferably at 50° C., however any temperature between 30° C. to 100° C. can also be used. Under these conditions DITC will covalently bind with primary amines such as those at the N-terminal of a peptide, thus the peptides will be covalently linked to the DITC glass. After incubation, the DITC glass is washed several times with organic and/or ionic buffers such as 50% acetonitrile or 1M salt in order to wash away all non-bound contaminating moieties. Finally the DITC glass is treated with an acidic solution such as neat trifluoroacetic acid (TFA) which causes the DITC to cyclize with the terminal amino acid of the bound polypeptide, thereby releasing the polypeptide minus its terminal amino acid, generating a released polypeptide of length n−1. Contaminants bound to the DITC glass will not be released by this process, thus separating them from the desired peptides, polypeptides, polymers or small organic molecules.

In a second embodiment of the invention the above procedure is performed with a centrifugal filter device (Microcon YM-3; Millipore, Bedford, Mass.) with a buffer resistant filter such as Durapore (Millipore, Bedford, Mass.). The sample/DITC glass reaction is performed in the upper chamber of the filter device with occasional gentle shaking. When the reaction is complete, the filter device is spun in a centrifuge causing the DITC glass to stay in the upper chamber while the liquid filters into the lower chamber where it can easily discarded. Washing of the DITC glass is performed in a similar manner. Finally, the release step is performed by addition of TFA to the DITC glass in the upper chamber. After incubation, the filter device is spun as before, however, the liquid in the lower chamber is kept and contains the purified peptides.

In a third embodiment of the invention, the above procedure is used to produce modified peptides and polypeptides for the differential analysis of two or more samples. In the final step of releasing the peptides or polypeptides bound to the DITC glass, an isotopically heavy acid such as deuterated TFA is used in lieu of TFA, which causes the peptides or polypeptides to be released with a single deuterated hydrogen at their N-terminus. In effect, each peptide or polypeptide is 1 Da (Dalton) heavier due to the deuterated hydrogen. When deuterated peptides or polypeptides are combined with a mixture of nondeuterated peptides or polypeptides, the two populations can easily be distinguished in a mass spectrometer such that a relative quantitation measurement between the nondeuterated peptide sample and the isotopically heavy peptide sample can be made.

One skilled in the art will recognize that a wide range of protein detection and measurement techniques, including sequencing, biological assay, peptide-bining small molecule drug screening, high performance liquid chromatography (HPLC), reversed phase HPLC, Fast Performance Liquid Chromatography (FPLC), electrophoresis, capillary electrophoresis and isoelectric focusing can be used to further characterize, measure or identify the purified peptides, polypeptides, polymers or small organic molecules. Mass spectrometric techniques also may be useful in the present invention. Representative examples of suitable spectrometric techniques include time-of-flight (TOF) mass spectrometry, quadrupole mass spectrometry, magnetic sector mass spectrometry and electric sector mass spectrometry. Specific embodiments of such techniques include ion-trap mass spectrometry, electrospray ionization (ESI) mass spectrometry, ion-spray mass spectrometry, liquid ionization mass spectrometry, atmospheric pressure ionization mass spectrometry, electron ionization mass spectrometry, fast atom bombard ionization mass spectrometry, MALDI mass spectrometry, photo-ionization time-of-flight mass spectrometry, laser droplet mass spectrometry, MALDI-TOF mass spectrometry, APCI mass spectrometry, nano-spray mass spectrometry, nebulised spray ionization mass spectrometry, chemical ionization mass spectrometry, resonance ionization mass spectrometry, secondary ionization mass spectrometry and thermospray mass spectrometry.

The present invention comprises a method for determining differential peptide and polypeptide expression patterns and levels between a first biological sample and a second biological sample. The method comprises providing a purified peptide mixture comprising peptides from a first biological sample and purified labeled peptides from a second biological sample, wherein peptides having the same amino acid sequence in the first biological sample and in the second biological sample have a predetermined mass difference; calculating the weight of peptides in the peptide mixture; identifying a peptide pair in the peptide mixture by determining two peptides whose weight differs by the predetermined mass difference; and quantifying the level of each peptide in the paired or differential samples. Preferably, the peptide mixtures are purified using the above described purification method of binding peptides, most preferably MHC-associated peptides, to ITC substrate, removing unbound material from the glass, and releasing the peptides from the ITC substrate by treating the bound peptides with an organic acid. The labeling of the peptides from the second biological sample and the mass differential is preferably achieved by using a deuterated acid to release the bound peptides from the ITC substrates.

The present invention comprises a method for quantitative proteomic analysis of two polypeptide populations. The method comprising: differentially labeling the two polypeptide populations by binding one of the polypeptide populations to ITC substrate and releasing the polypeptide population by means of a deuterated acid; combining the two polypeptide populations to form a mixed polypeptide population; proteolyzing the mixed polypeptide population to generate a collection of mixed peptide fragments of suitable size to be resolved by mass analysis; separating the collection of mixed polypeptide fragments by mass analysis into discrete peptide fragments while producing a primary mass spectrum with peptide peak intensities indicative of the presence of the discrete peptide fragments; analyzing the discrete peptide fragments using tandem mass analysis to generate a plurality of tandem mass spectrum characteristic of each discrete peptide fragment; comparing the tandem mass spectrum against a database of sequence-correlated mass spectra thereby determining a putative sequence identity of the peptide and its polypeptide of origin for the tandem mass spectrum generated by the discrete peptide fragments; identifying the discrete peptide fragments derived from the differentially labeled peptide populations which are indicative of analogous peptides; and assessing the peptide peak intensities of the discrete peptide fragments derived from the analogous peptides for the purpose of comparative analysis.

The present invention comprises a method of isotopically tagging a peptide and/or polypeptide specimen without altering biological properties thereof. The method comprises binding the peptides to a ITC substrate and subsequently releasing the peptides by reacting the bound peptides with a deuterated acid solution. Preferably, unbound contaminate moieties are removed prior to releasing the peptides from the ITC substrate.

The present invention provides a method for identification of polypeptides associated with Major Histocompatibility Complex (MHC) proteins affected by disease, for example viral infection or cancer. The method of identification comprises: (i) providing two test samples of MHC associated polypeptides isolated from two test cells, wherein one test sample is a reference sample and the other is a diseased sample and the polypeptides of each sample are separately isolated by binding the polypeptides of the samples to ITC substrate, removing unbound contaminate moieties, and applying an acid solution to release the polypeptides from the ITC substrate; (ii) by mass spectrometry using a quantitative mass analyzer, determining the levels of polypeptides in said test samples; (iii) comparing the composition and level of one or more of the polypeptides from said treated test sample with levels of respective polypeptides from said reference sample; (iv) identifying the sequences of polypeptides in said diseased sample which, relative to the reference sample, have altered abundance and/or altered levels of post-translational modification(s), thereby identifying the MHC-associated polypeptides affected by the disease.

In another aspect the present invention comprises a method for identifying a compound that alters the abundance of an MHC-associated polypeptide in a sample. The method comprises (i) providing a reference sample and a plurality of test samples of MHC-associated polypeptides, each isolated from a test cell treated by a specific test compound by separately extracting and binding the the polypeptides of each sample to ITC substrate, removing unbound contaminate moieties, and applying an acid solution to release the polypeptides from the ITC substrate; (ii) by mass spectrometry using a quantitative mass analyzer, determining the levels of said membrane-associated polypeptides in the test samples and the reference sample; (iii) comparing the level of one or more of said membrane-associated polypeptides from the test samples with levels of respective polypeptides from the reference sample; (iv) identifying the test sample which, relative to the reference sample, has altered abundance, thereby identifying the test compound responsible for the change.

In yet another aspect, the present invention provides method for identifying a compound that alters the levels of post-translational modification of a polypeptide in a sample. The method comprises (i) providing a reference sample and a plurality of test samples of polypeptides, each isolated from a test cell treated by a specific test compound by binding the polypeptides of the samples to ITC substrate, removing unbound contaminate moieties, and applying an acid solution to release the polypeptides from the ITC substrate; (ii) by mass spectrometry using a quantitative mass analyzer, determining the levels of said polypeptides in the test samples and the reference samples; (iii) comparing the level of one or more of said polypeptides from the test samples with levels of respective polypeptides from the reference sample; (iv) identifying the test sample which, relative to the reference sample, has altered levels of post-translational modification, thereby identifying the test compound responsible for the change.

In yet another aspect, the present invention provides method for identifying a disease state that alters the levels of post-translational modification of a polypeptide in a sample. The method comprises (i) providing a reference sample and a plurality of test samples of polypeptides, each isolated from a test cell treated by a specific test compound by binding the polypeptides of the samples to ITC substrate, removing unbound contaminate moieties, and applying an acid solution to release the polypeptides from the ITC substrate; (ii) by mass spectrometry using a quantitative mass analyzer, determining the levels of said polypeptides in the test samples and the reference samples; (iii) comparing the level of one or more of said polypeptides from the test samples with levels of respective polypeptides from the reference sample; (iv) identifying the test sample which, relative to the reference sample, has altered levels of post-translational modification, thereby identifying the changes brought about by the disease.

In yet another aspect, the invention provides for a method of purifying a biologically active compound such as small molecule drug. The method comprises binding the compound to the ITC substrate, removing unbound contaminating and unreacted moieties, and applying an acid solution to release the compound from the ITC substrate. Purity of the released compound can be assessed by any appropriate means such as chromatography or mass spectrometry.

In yet another aspect, the invention provides for a method for screening for small molecules that bind to peptides or polypeptides, for example, for identification of small molecule drugs that bind to specific protein or peptide targets. The method comprises (i) binding one or more peptides or polypeptides to an ITC substrate (ii) contacting the small molecule with the peptides or polypeptides bound to the ITC substrate (iii) washing away any contaminating moieties (iv) releasing bound compound from the ITC substrate (v) identifying the bound compound. Optionally, an acid solution may then be used to release the polypeptides from the ITC substrate.

The present invention provides a method of conducting a pharmaceutical business. The method comprises (i) by the above-described method, determining the identity of a target polypeptide isolated on the basis of the polypeptide being (a) having a differential cellular localization of interest; (b) having a differential expression pattern of interest; (c) having a differential post-translational modification of interest; or (d) having a differential abundance of interest; (ii) identifying compounds by their ability to alter the abundance or subcellular localization or post-translational modification of the target polypeptide; (iii) conducting therapeutic profiling of compounds identified in step (ii), or further analogs thereof, for efficacy and toxicity in animals; and, (iv) formulating a pharmaceutical preparation including one or more compounds identified in step (iii) as having an acceptable therapeutic profile. The business method may further comprise an additional step of establishing a distribution system for distributing the pharmaceutical preparation for sale and/or the step of establishing a sales group for marketing the pharmaceutical preparation.

The present invention provides a method of conducting a pharmaceutical business, comprising: (i) by the above-described method, determining the identity of a target polypeptide isolated on the basis of the polypeptide: (a) having a differential cellular localization of interest, (b) having a differential expression pattern of interest, (c) having a differential post-translational modification of interest, or (d) having a differential abundance of interest; (ii) (optionally) conducting therapeutic profiling of the target gene for efficacy and toxicity in animals; and (iii) licensing, to a third party, the rights for further drug development of inhibitors or activators of the target gene.

### EXAMPLES

The following examples illustrate embodiments of the methods and compositions of this invention. These examples are illustrative only, and do not limit the scope of the present invention.

### Example 1

### Extraction of Major Histocompatibility Complex (MHC) Molecules and Associated Peptides

Pan MHC class I molecules are isolated from aliquots of 1-5×10⁸ tumor and non-tumor cells after solubilization in buffer containing 1% NP-40 and a protease inhibitor cocktail. Affinity chromatography is performed using the monoclonal antibody W6/32, which selectively recognizes the MHC class I molecules. Bound material is eluted in 0.2N acetic acid, further acidified to 10% acetic acid, and boiled for 5 minutes. Low molecular weight peptides are separated from the HLA-A68 heavy chain, β₂ microglobulin (β₂m), and Ig heavy and light chains by ultrafiltration through a Millipore filter with a 5000 Dalton exclusion limit.

### Example 2

### First Round Fractionation of MHC-Associated Peptides by HPLC

Filtered samples undergo a first dimension fractionation via HPLC. A 1 mm×250 mm PLRP-S 100A° Polymer column is used with an Applied Biosystems Model 140B Separations System. Solvent A is 2% ACN in H₂O+0.1% TFA and solvent B is 80% ACN in H₂O+0.09% TFA using a gradient of 2% B to 60% B in 60 minutes at a flow rate of 50 ul/min. Column effluent is monitored at 214 nm and fractions are collected at one minute intervals.

### Example 3

### Isolation of MHC-Associated Peptides Using DITC Glass

Fractions are reduced to one half of their original volume using vacuum centrifugation without heat. They are then diluted to 100 microliters with 5% triethylamine in 50/50 isopropyl alcohol/water. Approximately 10 ug of isothiocyanate glass (DITC) is placed into a 0.1 um Durapore membrane spin filter for each fraction to be further analyzed. The fraction volume is added to the DITC in the spin filter and allowed to incubate for 45 minutes at 45° C. with gentle shaking. Only chemical molecules containing primary amines will react and covalently bind to the DITC derivatized glass. All filter units then undergo centrifugation at 10,000 rpm for 2 minutes. The filtrate volume is discarded. The DITC glass is then washed several times with organic and ionic buffers at neutral pH repeating the steps above: add buffer, shake, centrifuge, discard filtrate. Finally, the peptides are released from the DITC glass by the addition of 100 ul's of TFA, incubating for 10 minutes at 45° C. with gentle shaking. The filter units undergoes centrifugation at 10,000 rpm for 2 minutes and the filtrate containing peptides is saved.

### Example 4

### Differential End-Labeling of Peptides with Deuterated TFA

Differential analysis of peptides from different sample is achieved by modifying the group of peptides that correspond to the differential sample. The tumor sample from Example 1 is processed as described in Examples 1 through 3. The differential peptide extract is released from the DITC glass using deuterated TFA, thus increasing its molecular weight by 1 Dalton which can be easily seen in a mass spectrometer.

### Example 5

### Combination of Tumor and Non-Tumor Samples for Differential Analysis

The deuterated sample of Example 4 and non-deuterated sample of Example 3 are combined together into one solution and undergo further analysis as a mixture. The mixture is analyzed using a mass spectrometer.

All references cited above are incorporated herein by reference. Numerous modifications and variations of the present invention are included in the above-identified specification and are expected to be obvious to one of skill in the art. Such modifications and alterations to the compositions and processes of the present invention are believed to be encompassed in the scope of the claims appended hereto:

1) A method for the purification and quantitative recovery of a polypeptide of length n having a free amino terminus comprising the steps of:
a) attaching said polypeptide to a solid support derivatized with isothiocyanate chemistry via the amino terminus of said polypeptide; and
b) releasing said polypeptide from the solid support by cleavage of the terminal amino acid of said polypeptide with organic acid to produce a polypeptide of length n−1.

2) A method of labeling a polypeptide of length n having an amino terminus comprising the steps of:
a) attaching said polypeptide to a solid support derivatized with isothiocyanato chemistry via the amino terminus of said polypeptide; and
b) releasing said polypeptide from the solid support by cleavage of the terminal amino acid of said polypeptide with isotopically labeled organic acid and incorporating the label at the N terminus of a polypeptide of length of n−1.

3) A method for identification of MHC-associated polypeptide targets of a compound, comprising: (i) providing two test samples of MHC-associated polypeptides isolated from two test cells, wherein one test sample is a reference sample and the other is a sample treated by said compound, and the polypeptides of each sample are isolated by binding the polypeptides of the samples to ITC substrate, removing unbound contaminate moieties, and applying an acid solution to release the polypeptides from the ITC substrate; (ii) by mass spectrometry using a quantitative mass analyzer, determining the levels of polypeptides in said test samples; (iii) comparing the level of one or more of the polypeptides from said treated test sample with levels of respective polypeptides from said reference sample; (iv) identifying the sequences of polypeptides in said treated sample which, relative to the reference sample, have altered abundance and/or altered levels of post-translational modification, thereby identifying the MHC-associated polypeptide targets of said compound.
